(19) United States
(12) Patent Application Publication
(10) Pub. No.: US 2004/0105074 A1
(43) Pub. Date: Jun. 3, 2004

(54) Digital stereo image analyzer for automated analyses of human retinopathy

(76) Inventors: Peter Soliz, Albuquerque, NM (US); Sunanda Mitra, Lubbock, TX (US); Mark Wilson, Albuquerque, NM (US); Enrique Corona, Pueble (MX)

Correspondence Address: ROBERTS ABOKHAIR & MARDULA, SUITE 1000, 11800 SUNRISE VALLEY DRIVE, RESTON, VA, 20191, US

(21) Appl. No.: 10/631,373

(22) Filed: Jul. 31, 2003

Related U.S. Application Data
(60) Provisional application No. 60/400,964, filed on Aug. 2, 2002.

(51) Int. Cl. A61B 3/14

(52) U.S. Cl. 351/206

(57) ABSTRACT

A system and method of automated analysis of human retinopathy. Stereo images of a retina are obtained at different times. Topography of the retina is obtained from analysis of the stereo image pair. Cupping of the retina is then ascertained and is indicative of the progression and severity of various retinopathies. Disparity mapping of the retina from the various images are also obtained from the images and compared at different times to assess how retinal disparities are changing as yet another indicator of the progression of retinopathies.

### CROSS REFERENCE TO RELATED APPLICATION

This application claims benefits under 35 U.S.C. § 119(e) of provisional application No. 60/400,964 filed Aug. 2, 2002, which incorporated by reference herein, in its entirety, for all purposes.

### STATEMENT OF GOVERNMENT INTEREST

This research has been partially supported by funds from the Texas Technology Development and Transfer (TDT) Program (Grant #003644-0217-2001) the NEI (Grant #1 R43 EY14090-01) and the NSF (Grant EIA-9980296).

### FIELD OF THE INVENTION

This invention relates generally to the field of stereo image analysis. More particularly the present invention provides a digital stereo vision system and method for surface recovery of the topography of the optic nerve head (ONH) from stereo pairs of optic disc images.

### BACKGROUND

Optic disc cupping, which is most frequently seen with glaucomatous optic atrophy, may be due to a number of disorders that affect the optic nerve. Some investigators have suggested that ischemic optic neuropathy from giant cell arteritis (GCA), known as arteritic anterior ischemic optic neuropathy (AAION), may be an important cause of optic disc cupping, which can mimic that seen in glaucoma. Thus optic disc cupping may be indicative or a variety of retinopathies.

In the case of glaucoma, an elevated intraocular pressure is often associated with the disease. Current literature indicates that the measurement of intraocular pressure cannot be a reliable predictor of visual function loss from glaucoma. Early detection of glaucoma is particularly significant since it allows timely treatment to prevent major visual field loss and prolongs the effective years of usable vision. The major limitation of precise evaluation of early glaucomatous changes in present clinical situations still remains the inability of the human vision system to detect subtle changes and make precise estimates of size, shape, and color of pathological features.

Change in cupping of the optic disc represents a valuable indicator for the ophthalmologists to diagnose and monitor the disease. The requirement for a low cost, easy to use, and widely accepted diagnostic measure for glaucoma is met in most clinics principally by standard photography, either with a stereo camera that collects the image pair simultaneously or through the use of standard retinal imaging cameras that collect the image pair sequentially. Accurate measures of the optic nerve head are crucial in the early diagnosis and follow-up management of chronic glaucoma since glaucoma can cause damage in the optic nerve and ultimately loss of vision.

Currently, the condition of the ONH is qualitatively evaluated by observation of a pair of stereoscopic fundus photographs by an ophthalmologist in addition to measurements of cup to disc ratios from the same photographs. Early diagnosis of glaucoma is based on observations of experienced clinical observers and manual drawing of the contours of the ONH, and thus the procedure required is tedious and time-consuming, and most of all prone to variations in their interpretation that may mask subtle changes due to the disease progression.

Retinal diseases such as diabetic retinopathy, age related macular degeneration (ARMD) and glaucoma are common causes of early visual loss and blindness. Early detection of diabetic retinopathy and glaucoma is particularly significant since it allows timely treatment to prevent major visual field loss and prolongs the effective years of usable vision. Therefore, development of precise as well as automated methods of clinical measures for evaluating changes in retinal features, such as the ONH topography in glaucoma, is essential.

Because the ophthalmoscope is still the most widely used instrument employed by clinicians to diagnose retinal diseases, the diagnostic information from its use and the interpretation of retinal images from funduscopes are dependent on the expertise of the clinician.

The major limitation to precise evaluation of early retinopathy in present clinical situations still remains the inability of the human vision system to detect subtle changes and make precise estimates of size, shape, and color of pathological features. This limitation leads to intra- as well as inter-observer variability in sensitivity and specificity. The subjective evaluation and interpretation of the optic nerve head have been reported with documentation of inter- and intra-observer variations. Zangwill et al. has developed a quantitative grading procedure for measuring cupping, defined by cup contour, for use in population based studies.

To overcome the human vision limitations, a number of advanced retinal imaging systems have been developed over the years for early detection of glaucoma. Ophthalmic systems using laser scanning and optical interferometric techniques have been designed to detect nerve fiber layer (NFL) loss in glaucoma. However, the clinical utility of the above mentioned quantitative approaches have not been shown to be superior to expert interpretation of stereo disc photography, and the latter is still the technique commonly employed in clinical settings for documenting optic disc changes.

Digital stereo image interpretation in clinics by physicians, to derive 2-D measures such as the optic cup/disc ratios, is not yet fully automated. The need for human intervention continues to be a principal source of variability. The major limitations of precise evaluation of retinal structures in present clinical situations are the lack of standardization, the inherent subjectivity involved in the interpretation of retinal images by physicians in a clinical setting, and intra- as well as inter-observer variability in sensitivity and specificity.

In summary, detection of the changes in retinal structures is an important metric for evaluating the onset or progression of ocular diseases. Optic cup/disc and blood vessel shape changes caused by retinopathies are currently assessed through manual, time consuming methodologies with inherent subject variability. Automated detection of these shape changes requires the segmentation of specific features and contours during the preprocessing stages. What would be particularly useful is a more automated way to recovery the topography of the retina and measure the cupping that occurs in an optic disc as an indicator of retinopathies.

### SUMMARY OF THE INVENTION

The condition of optic nerve head (ONH) for the detection of glaucoma and other diseases is traditionally evaluated by manual observation of a pair of stereographic images and subsequent drawing of cup and disc contours. Automated methods attempt to use the same stereographic images to find the disparity between pixels in order to form a three-dimensional map of the optic cup/disc. Disparity calculations can be obtained through pyramidal surface-matching algorithms based on optimum surface recovery within a 3-D cross-correlation coefficient volume. Fully automated methods for calculating disparity can be problematic and fail due to the problems of noise, occlusion, and distortions, and the success of these disparity algorithms are therefore extremely dependent on the initial feature extraction process. Because of this the present invention uses preprocessing and feature extractions to create a disparity map of the retina. The present invention provides a system and method for creating disparity maps from fundus images as more fully set forth below.

It is, therefore, an object of the present invention to provide an improved 2-D measure of the optic cup to optic disc ratio.

It is another object of the present invention to decrease the time required to obtain a 2-D contour of the optic disc.

It is yet another object of the present invention to provide a method for evaluating the effect of drug therapy on the eye.

It is a further object of the present invention to provide a standardized interpretation of retinal images by health care providers.

It is another object of the present invention to improve detection of subtle changes in the optic disc.

It is yet another object of the present invention to provide automated method for measuring changes in retinal features.

It is a further object of the present invention to provide a low cost diagnostic measure for glaucoma.

It is yet another object of the present invention to provide an accurate method to measure optic nerve head.

A further object of the present invention to provide a method to early detection of glaucoma.

The present invention employs a combination of power cepstrum and zero-mean normalized cross correlation (ZNCC) techniques embedded within a coarse-to-fine disparity search algorithm that extracts depth information from disparity between corresponding windows. The resulting sparse disparity matrix elements are encoded as gray levels and processed through a cubic B-spline operation to reduce intrinsic noise and generate a smooth representation of the optic disc/cup surfaces and 3-D measures from computer generated optic disc/cup volumes. The present invention improves longitudinal detection of subtle changes in the optic disc that may be difficult or impossible for ophthalmologists to detect and to quantify.

Additional objects and advantages of the present invention will be apparent in the following detailed description read in conjunction with the accompanying drawing figures.

### BRIEF DESCRIPTION OF THE DRAWINGS

FIG. 1 illustrates a schematic of a non-convergent visual system

FIG. 2 illustrates a schematic block diagram of the analysis and model in involved for 3-D visualization of a stereo image pair.

FIG. 3 illustrates a plot of ZNCC coefficients versus angle of rotation.

FIG. 4 illustrates compensation for translation and rotation within the stereo pair of images.

FIG. 5 illustrates a feature extrapolation process.

FIG. 6 illustrates disparity search process and interpolation.

FIG. 7 illustrates a final 3-D representation of the ONH.

FIG. 8 illustrates disparity distribution of a glaucoma patient.

FIG. 9 illustrates the difference between manual and semi-automatic segmentation images.

FIG. 10 illustrates cup/disc ratios versus year of study for patient one.

FIG. 11 illustrates a longitudinal study of cup/disc ratios for three patients.

### DETAILED DESCRIPTION

The present invention employs a combination of power cepstrum and zero-mean normalized cross correlation techniques embedded within a coarse-to-fine algorithm that extracts depth information from disparity between corresponding windows. The power cepstrum is the inverse Fourier transform of the logarithm of the power spectrum of a signal. The resulting sparse disparity matrix elements are encoded as gray levels and processed through a cubic B-spline operation to reduce intrinsic noise and generate a smooth representation of the optic disc/cup surfaces and 3-D measures from computer generated optic disc/cup volumes. The present invention improves longitudinal detection of subtle changes in the optic disc that may be difficult or impossible for a human observer to such as an ophthalmologist or other medical professional to detect and to quantify.

Currently, the condition of the ONH is qualitatively evaluated by observation of a pair of stereoscopic fundus photographs by an ophthalmologist in addition to measurements of cup to disc ratios from the same photographs. Early diagnosis of glaucoma is based on observations of experienced clinical observers and manual drawing of the contours of the ONH, and thus the procedure required is tedious and time-consuming, and most of all prone to variations in their interpretation that may mask subtle changes due to the disease progression.

Fundus image enhancement and stereo image analysis based on advanced signal/image processing methodologies such as cepstrum analysis for registration of stereo images, and disparity to depth mappings and 3-D surface recovery of the optic disc using cubic B-spline interpolation is the basis of the present invention. The present invention is also directed to an automated computerized technique for 3-D measures of the cup/disc ratios of the ONH from stereoscopic pairs of fundus images based on advanced image analysis techniques involving 3-D surface recovery from stereo disparity and registration using Fourier methods.

The present invention automates 3-D ONH measures in order to provide consistent and improved evaluation and follow-up of glaucomatous ONHs. The present invention yields quantitative evaluation of deformation in the ONH in terms of additional measures such as the change in the volume of the cup/disc in a longitudinal follow-up study of a patient.

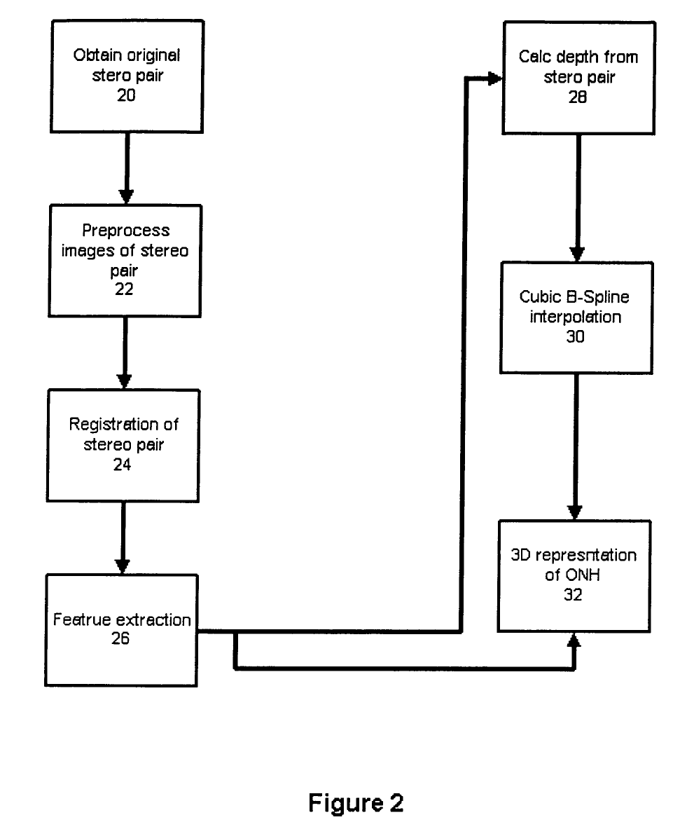

In one embodiment, the present invention is a system and method to derive a three-dimensional surface from two-dimensional stereo data. Several stages are involved including preprocessing and initial registration of the stereo pair, disparity finding, and interpolation of the sparse disparity maps. Two cameras capture the same 3-D real world image from different perspectives, providing a pair of stereo images. The coordinate associated with depth of this scene can be extracted by triangulation FIG. 2, 28 of corresponding points in the stereoscopic images. In the process of finding disparities between conjugate pairs of points, image-matching strategies are employed.

According to the matching strategy used (to find these disparities) the processes of searching can be either area based or feature based. Area based strategies intend to match image areas, while feature based processes try to match whatever feature seems to be in the stereo pair. An area based matching technique using ZNCC (zero-mean normalized cross correlation) as the disparity measure is employed in the present invention. The disparity measures employed in the present invention involved ZNCC that is expressed as follows:

$$\begin{matrix}C ( i , j ) = \frac{{cov}_{i , j} ( f , g )}{{σ}_{i , j} ( f ) \times {σ}_{i , j} ( g )} , & 1 ) \\ {cov}_{i , j} ( f , g ) = {\sum }_{m = i - K}^{i + K} {\sum }_{n = j - L}^{j + L} ( {f}_{m , n} - \overline{{f}_{i , j}} ) ( {g}_{m , n} - \overline{{g}_{i , j}} ) , & 2 )\end{matrix}$$

where ƒ and g are the windows of pixels to be measured. K and L define the size of those windows, and the indices for the pixels within the windows are i and j. σ(ƒ) and σ(g) are the square roots of the covariances, cov(ƒ,ƒ) and cov(g,g) respectively. Highest coefficient shows the amount of degrees that must be used for rotational compensation

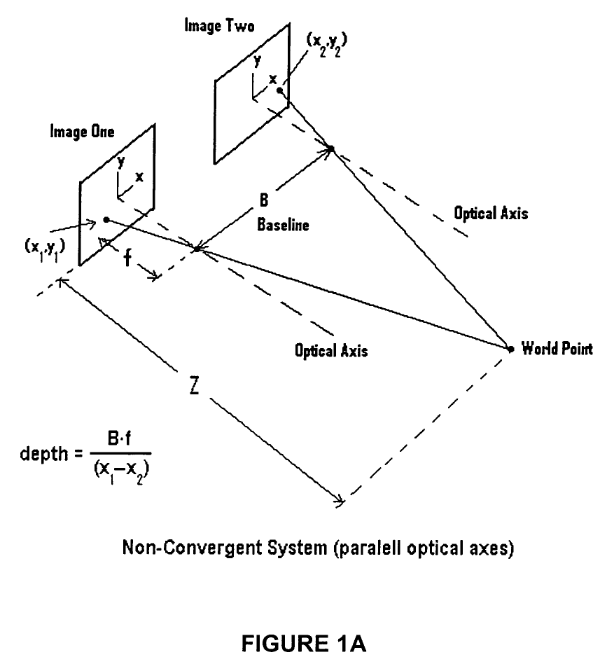

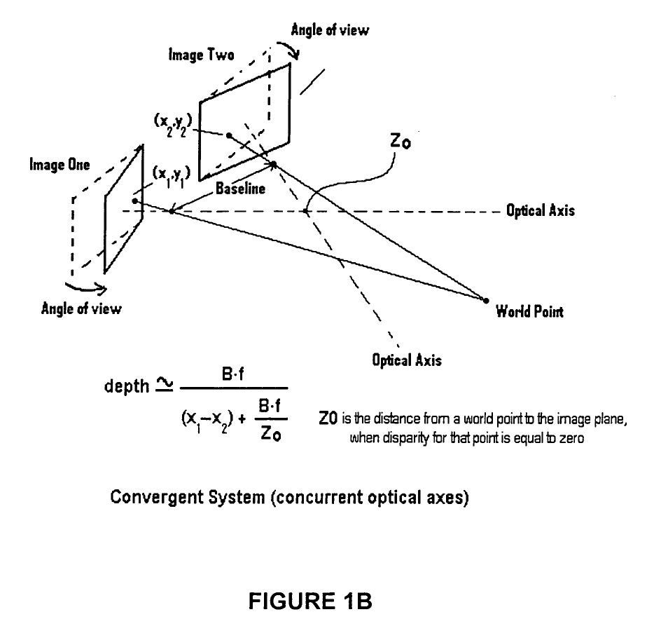

Referring first to FIG. 1 Convergent and non-convergent optical systems are illustrated. These are the two primary vision systems for implementing stereo vision. In the non-convergent system, which better approximates the clinical imaging situation, the disparity is inversely proportional to depth. In the convergent system, the disparity between corresponding points can also be shown to be inversely proportional to the depth, however, some other parameters are involved as well. FIG. 1 shows the convergent and non-convergent systems and their relationships with the calculated disparities in the stereo pair.

The present invention uses a non-convergent system as a good approximation to the clinical imaging situation and uses both feature and area based matching techniques to compute disparity between conjugate pairs of the same optic disc. However, this geometry is not meant as a limitation. Any imaging system that provides appropriate stereo imagery from which calculation can be made is appropriate for the present invention.

The stereo disc photographs used in accordance with one embodiment of the present invention are taken with a Zeiss fundus camera (Carl Zeiss, Thornwood, N.Y.) on Kodachrome 25 or Ektrachrome 100 film (i.e. color image). Other equipment and film or magnetic or optical recording media are also appropriate and known in the art. Stereopsis was achieved by decentration of the camera angle. Results presented herein are generated by using color photographs that are digitized using a Nikon LS-2000 slide scanner. The original slides were cropped to 15-degree fields of view during the scanning process to produce 512×512 pixel images saved as TIFF files. Although the initial inputs to the digital stereo image analyzer are color images, extraction of binary features (blood vessels) is necessary for better cepstral matching. As noted above, the term cepstrum has come to be accepted terminology for the inverse Fourier transform of the logarithm of the power spectrum of a signal

In one embodiment, the power cepstrum uses a matching technique where the frequency of the signal is greater than or equal to the frequency of the noise present, thus requiring sharp edge features (binary features) for satisfactory performance. However, when pyramidal-structured correlation coefficients between the stereo pair pixels are computed, windows without large featureless regions are used for finer disparity search. A constraint applied by the vision model used is that no disparity is expected on the vertical axis (only horizontal shifts are allowed). To assure this, the pair of images for which disparity is to be calculated is, first, vertically registered. This registration combines power cepstrum and frequency spectrum analysis to compensate for unwanted rotations and shifts that exist within the stereo pair.

At a given level of coarseness, the pair of images is divided into square windows of a given size according to the current level of coarseness. For corresponding windows in the stereo pair, the power cepstrum of the sum of both windows is calculated, as well as the cross-correlation (using zero-mean normalized cross correlation or ZNCC) along a range of pixels varying from minus one half of the window size to plus one half of the window size.

From the set of possible horizontally shifted pixel positions obtained by cepstral analysis, the one with the highest correlation coefficient is considered to be the disparity associated with each element in the whole window. After every disparity has been calculated for each pixel in the pair, a new stereo pair is generated consisting of the same size windows as the old stereo pair but shifted by the number of pixels in the previous window. Then, the search window size is halved and the search is performed again on the new stereo pair.

When the window size is reduced to the size of 8 by 8, a low pass filter is applied to every matrix calculated at each resolution stage and are added to get a resulting sparse disparity matrix. These data are fed into a cubic B-spline interpolation FIG. 2, 30 routine that smoothes the computed disparity matrix. Then features such as blood vessels are superimposed to help as landmarks in the final 3-D representation of the ONH. It should be noted that the 8 by 8 window is exemplary only and is not meant as a limitation.

Computer generated measures such as cup to disc ratios in volume and area can then be calculated from these data by segmenting the cup and disc contours from iso-disparity contours generated at each depth. FIG. 2 illustrates the overall process for the 3-D visualization of the stereo image pair. Although the registration and the disparity search processes are fully automated, the cup and disc contours can be interactively adjusted from the iso-disparity contours for better matching with the contours manually generated by the ophthalmologists.

An original stereo pair is obtained 20. For preprocessing and stereo pair registration, three channel (RGB) decomposition is performed on the original color pair 22. In one embodiment, only the green channel is processed since it is the one that carries the most information. Red and blue channels have low entropy in relation to the green channel and therefore are not taken into account. A registration process 24 removes all vertical displacements leaving only the horizontal shifts arising from the different positions of the camera while taking the stereo fundus images

In one embodiment of the present invention a power cepstrum based registration is employed that uses Fourier spectrum properties to correct rotational errors that may be present in the stereo pair. This process begins by extracting the most relevant features such as the blood vessels in both images. These features are extracted by subtracting a filtered version of the original stereo pair from the original (unsharp masking). After rendering this new stereo pair in a binary form, multiple passes of a median filter are used to eliminate some of the resulting noise in the images.

Feature extraction 26, then occurs as more fully set forth below. These features serve as “landmarks” for superimposition on the final 3-D image that is created (below) Compensation for rotational differences is also performed via ZNCC correlation of the Fourier spectrum of the images. According to the inherent Fourier spectrum properties, a rotation in the spatial image results in the same amount of rotation of its spectrum. Thus it is possible to find the angle of rotation of one image in the stereo pair with respect to the other by performing step-by-step rotations and cross correlating their Fourier transforms. The actual angle of rotation will be the one with the highest cross-correlation obtained.

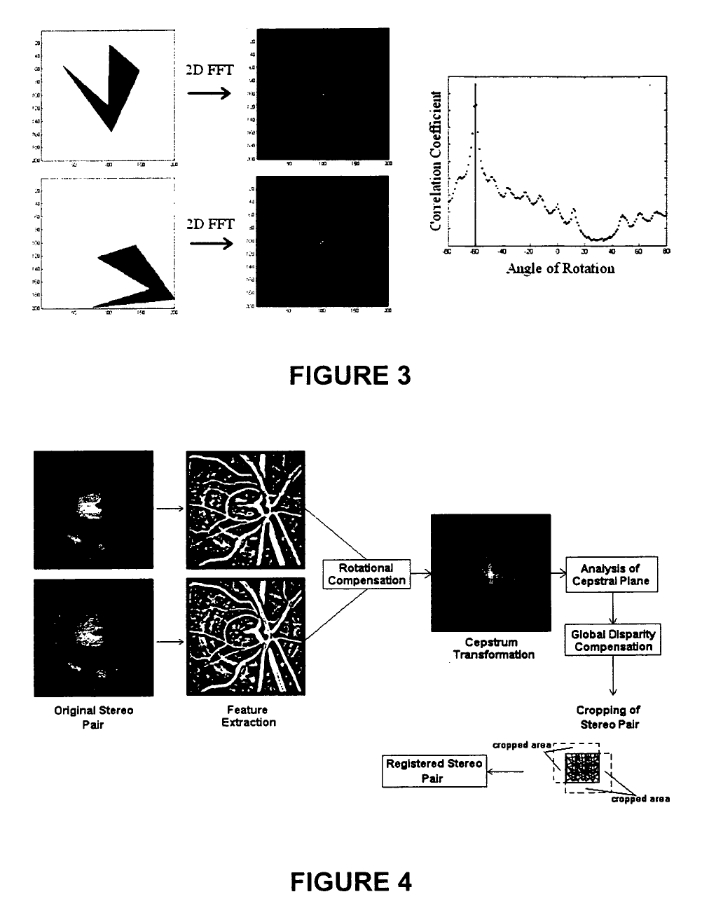

Rotational compensation is applied once the angle of rotation has been found. FIG. 3 shows an example plot of ZNCC coefficients at each angle step for which the search was performed. In the examples provided, the search is performed from a range of −2 to +2 degrees in 0.1 degree steps.

After the rotational correction, a cepstrum transformation is applied to the sum of the binary-featured stereo pair images. The power cepstrum P is defined as in [14]:

P[i(x, y)]=|F(1n{|F[i(x, y)]|²})|², 3)

where F represents the Fourier transform operation. Let w(x,y) be the reference image, w(x+x₀,y+y₀) be the shifted image and i(x,y)=w(x,y)+w(x+x₀,y+y₀). Then the power cepstrum of the sum of both images is given as:

P[i(x, y)]=P[w(x, y)]+Aδ(x, y)+Bδ(x±x₀, y±y₀)+Cδ(x±2x₀, y±2y₀)+ . . . , 4)

where δ(x,y) is the Kronecker delta and A, B, C are the first three coefficients for this power cepstrum expansion series.

Equation (4) shows that the displacement between images results in the sum of the power cepstrum of the original image w(x,y) plus a multitude of delta functions. Each delta is separated from the others by an integer multiple of the actual displacement being sought. In order to enhance the cepstral peaks, the cepstrum of the reference must be subtracted from the cepstrum of the stereo pair.

A fixed number of deltas are chosen from the resulting cepstrum. Each delta represents a translational shift, or an integer multiple of the shift, of a pixel in the shifted image from the corresponding pixel in the reference image. All points are tested by cross correlating the reference image with the other image shifted by the number of pixels (in the vertical and horizontal directions) indicated by the current point being tested.

The highest correlation will correspond to the most probable relative translation between both images. Once the translation of one image with respect to the other is known, compensation is performed. Some cropping is necessary after compensation to get rid of regions with no common information. After compensation of these displacements and cropping, the stereo pair is ready for further processing. The registration process is shown in FIG. 4.

In one embodiment, the present invention preprocesses the green channel of the multi-spectral stereo fundus images to extract salient features in the images. The same unsharp masking procedure as in the previous registration stage is used again. This increases the contrast level in the high frequencies (edges) that can be segmented later by thresholding. Although the green channel is chosen here (because of better contrast in this channel rather than in the blue or red), a gray scale conversion taken directly from the color stereo pair can also be used without noticeable loss of detail in the binary (feature) images.

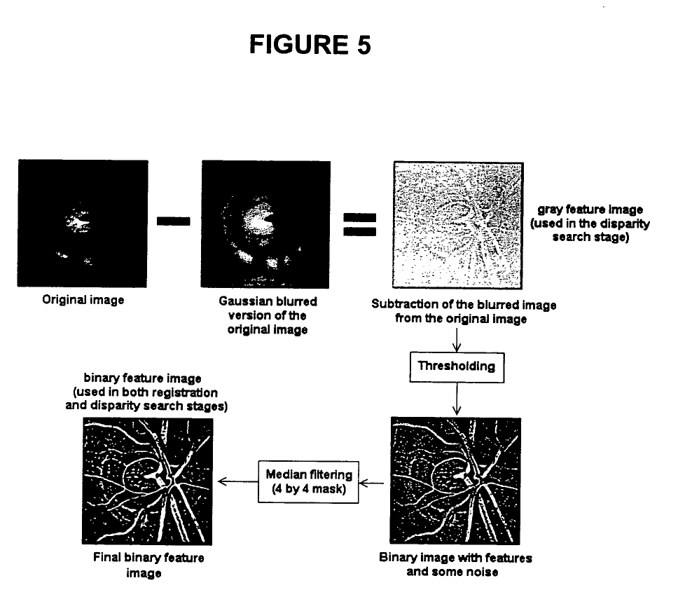

After thresholding the stereo pair, blood vessels are segmented along with some noise intrinsic to the unsharp masking method used. To filter out some of the remaining noise, a median filter is applied to get a clearer binary representation of the features. FIG. 5 shows the steps followed by the binary feature extraction technique. It is important to mention that a combination of binary and gray scale features is used in the disparity search stage. The gray scale features are obtained using the unsharp masking as done with the binary features, but no thresholding is performed. Since both the power cepstrum and ZNCC are used to find disparities, the feature stereo pair must have characteristics that favor both methods. Cepstrum performs well as long as the bandwidth of the noise remains below the bandwidth of the signal, showing the necessity for sharp edges, which can be provided by a binary image. The correlation coefficient method works well as long as there are no large flat regions. This is the reason for including the unsharp masked gray image also. The superposition of the binary image over the gray scale image will be the final feature image from which disparities are calculated

The present invention receives the feature stereo pair and outputs a disparity map showing right and left displacements between corresponding points. At this point, only horizontal shifts are expected between the two images in the stereo pair. The present invention divides both images into square windows of a given size (multiple of two), for example N by N. Cross-correlation (ZNCC) is performed between the windows in one image with the windows in the other image.

If cross-correlation is larger than a certain threshold, it is assumed that the windows at that position in the image are similar, so the cepstrum is applied to those windows to check for possible shifts. Otherwise, if the cross-correlation is smaller than or equal to the threshold mentioned, zero disparity is assigned to every pixel in the window. Only a specified number of horizontal points shown in the cepstrum are taken into account for analysis. For example, for an N by N window, only N/4 horizontal points are chosen for analysis in the cepstral plane. This is because for an N by N window the maximum horizontal displacement that can be detected is N/2 (either to the right or to the left, making a full range of N pixels), so checking all N/2 points for right and left shifts will be very time consuming. Instead, only the most probable N/4 horizontal shifts found by the cepstrum will be tested using the cross-correlation technique.

One of the images of the stereo pair is considered as the reference image and the other is the test image. Then, for every point chosen (from the cepstrum), cross-correlation is applied between the reference window (in the reference image) and the other window (in the test image) shifted by the number of pixels determined by the cepstral shift. Since the cepstrum can only detect the amount of the shifts but not their direction (the cepstrum is symmetric about the origin), each point should be tested for left and right shifts. So when checking N/4 cepstral points, actually N/2 positions are analyzed. The highest value in the cross-correlation will be the most probable shift that will be assigned to all elements in the window currently being tested for disparity. The number of cepstral points is not a fixed parameter and can be modified. This modification will affect the processing time and the accuracy of the disparity map.

Once all disparities have been calculated with a window size of N by N pixels, the size of the window is reduced by a factor of two and the whole process is repeated until the windows reach a predetermined size. Each disparity map (calculated at a given resolution) is accumulated by adding it to the previous disparity map.

At the end of the process, the final disparity map is the total accumulated disparity map. Usually the starting window size is 64 by 64 and the stopping size is 8 by 8. Smaller sizes of a window may not be worth computing because of the much longer time required and the small impact of it on the final disparity map. Also, since the window is so small, chances are that noise becomes a serious issue.

The cepstrum is a noise tolerant technique that is suitable for finding disparities in chosen regions while cross-correlation is noise sensitive and finds disparities using a procedure in a pixel-by-pixel fashion. The present invention combines both techniques resulting in an accurate and noise tolerant analysis method for generating 3-D representation from a stereo pair of images.

In the first coarseness level, disparities found to be equal to one half of the size of the window (either positive or negative) are checked in an alternative way. Such a procedure is followed since the disparity reported on the boundary of the search may be only an indication that the window is too small to catch a larger disparity. In this specific case illustrated herein, the disparity (for that specific window) is calculated as an average of the neighboring windows.

In order to obtain an accurate 3-D representation from a stereo pair of images, disparities should be known for each point (pixel) of one image with respect to the other. Since the disparity search algorithm only finds disparities for the features or regions, disparities of all individual pixels are not known. The interpolation used here gives an estimate of the other missing disparities. Cubic B-spline interpolation technique FIG. 2, 30 applied to the sparse matrix resulting from the disparity search. The cubic B-spline can be modeled by three successive convolutions with a constant mask. In this case, a mask consisting of all ones of size 32 by 32 or 64 by 64 is used. After filtering the original sparse disparity matrix three times with the mask described above, a smooth representation results. This is the final 3-D surface of the ONH FIG. 2, 32. With this surface, measures such as the disc and cup volume can be made. Further, the features extracted in the process of FIG. 2, 26 are superimposed on the 3-D surface created FIG. 2, 32.

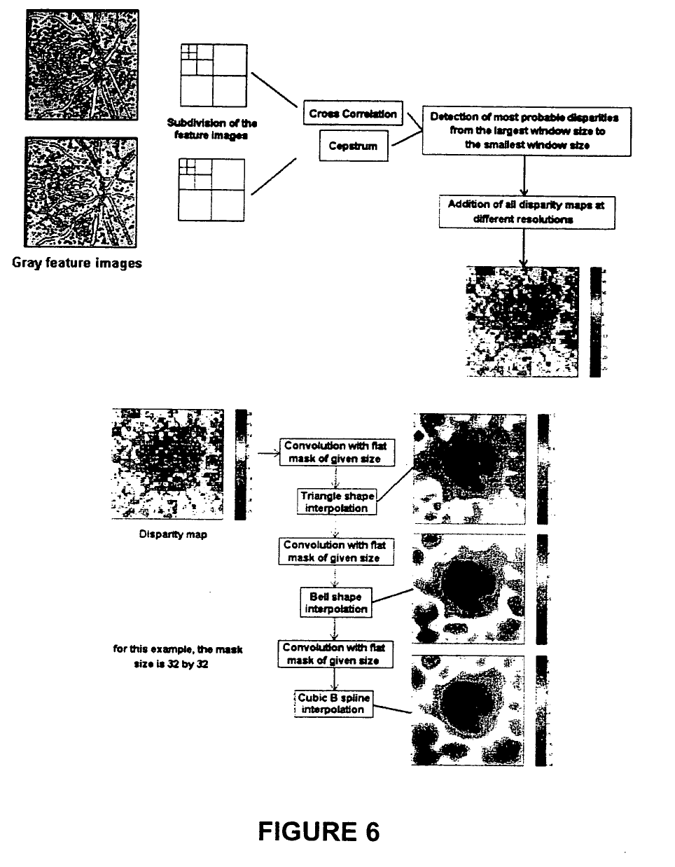

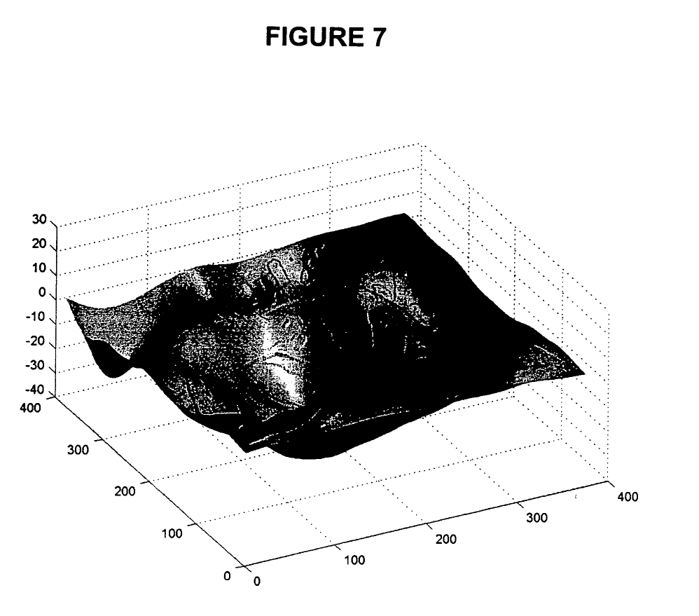

A disparity search process and the interpolation are shown in FIG. 6. In this figure, only for illustrative purposes, the three convolutions are shown. The actual implementation is performed in the frequency domain by multiplying the Fourier transform of the flat kernel mask by itself three times. This kernel mask is then padded with zeros to fit the size of the disparity map. The Fourier transform of the disparity map is taken and multiplied by the padded kernel. Finally, an inverse Fourier transform is taken for mapping the data back to the spatial domain. A contrast reversed, linearly stretched feature image is superimposed on the depth image in order to visualize the blood vessels as landmarks on the 3-D representation of the ONH. This step is very important for ophthalmologists because longitudinal data can be more easily analyzed when reference points can be distinguished on ONH 3-D representations. A mask of the same size as the image is constructed from one of the images in the binary stereo feature pair. This mask is multiplied by the original gray scale image, leaving only blood vessels along with some noise that can be filtered out by thresholding. Once this is done, the resulting picture is added to the final 3-D surface of the ONH. This is the final the 3-D representation of the ONH. FIG. 7 shows the final representation obtained with the methodology described previously.

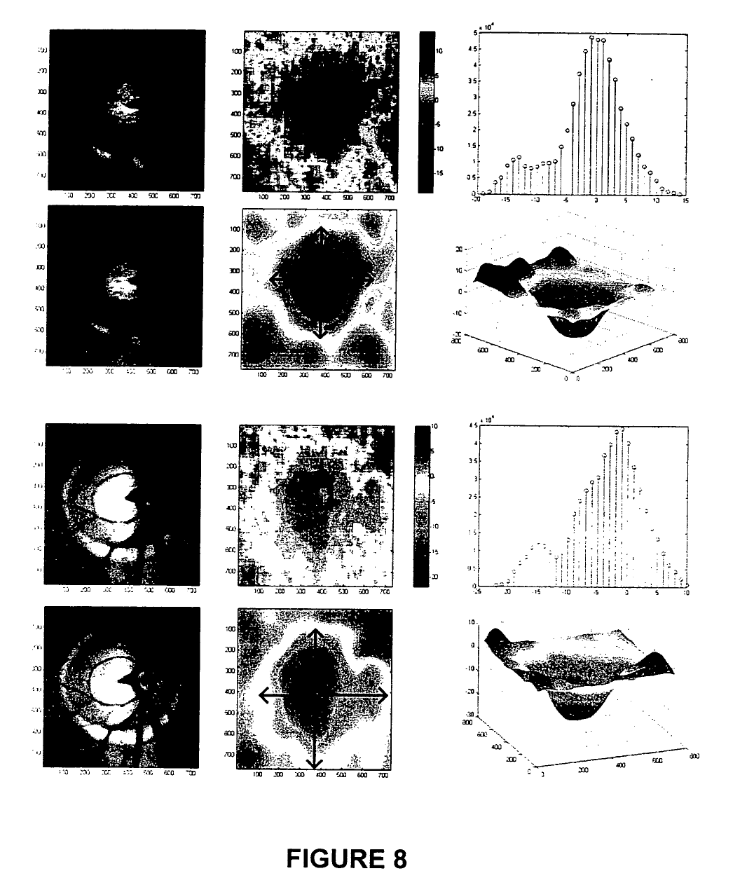

FIG. 8 is an example of a 2-D disparity map. The disparity distributions and the corresponding 2-D disparity maps are shown in color for the same eye of a glaucoma patient from stereo disc photographs taken in 1994 on the top and the same taken in 1999 on the bottom. Further spreading of vertical and horizontal elongations of the disparity maps through the years suggests the progression of the disease. Disparity distribution of a glaucoma patient was obtained from fundus images. The disparity distribution from the same eye of the same patient taken in 1999 is shown on the bottom. Disparity maps appear in color although this is not a limitation. Any graphical representation technique may be used to show this result. A change in volume indicates progression of glaucoma.

Results

The semi-automatic segmentation method for the cup and disc of the present invention comprises manually looking for the iso-disparity contours from the automatically generated smoothed disparity matrices that better enclose the cup and disc. In this context, the best contour is the one that has more points in common with the manual segmentation from the ophthalmologist. Prior to the present invention, initial results were evaluated by semi-automatic segmentation of the cup and disc using the interpolated version of the disparity maps found in the previous stages. There is also a manual segmentation performed by a group of trained ophthalmologists.

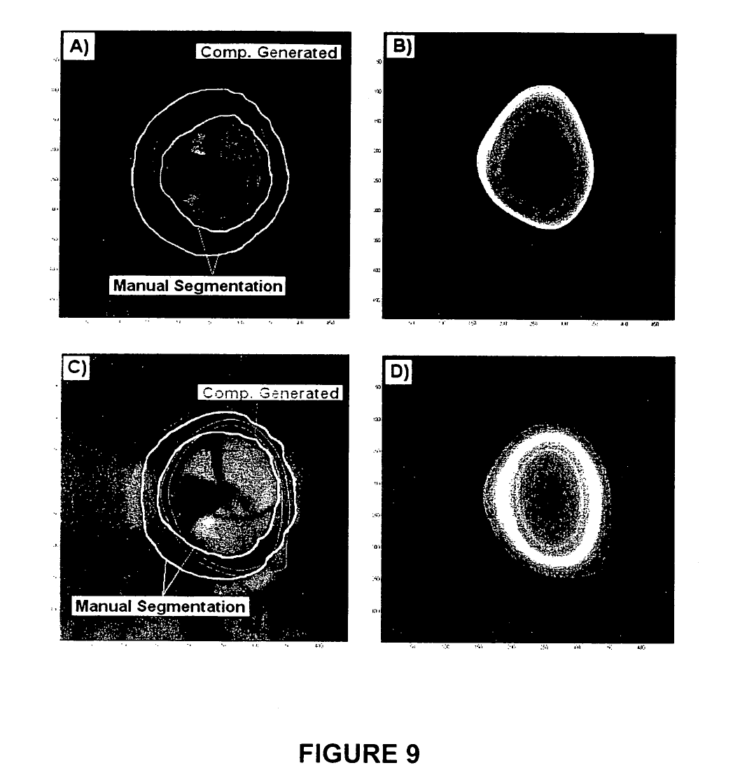

The computer generated measures are compared with manual measures used by the ophthalmologists for a test data set of longitudinal stereo fundus images of glaucoma patients spanning over 20 years to determine the validity of computer generated 2-D and 3-D cup/disc measures in monitoring progression of glaucoma. In FIG. 9, both segmentations (clinical and semi-automatic) are shown for the same patient with glaucoma. The stereo pairs were taken in 1989 and 1999. Notice that the contours are not exactly the same but they have similar shapes. Segmented cup and disc (both computer generated and ophthalmologist segmentation) are used to obtain volume measures from the 3-D ONH final representation. The cup volume is that contained in the portion of the cup within the depth representation. The disc volume is obtained in the same manner. The cup (or disc) volume is found simply by accumulating the intensity values on the depth map enclosed in the segmented cup (or disc). If more precision is needed, subpixel interpolation can be performed, thus increasing the number of intensity values to be accumulated within the disc or cup segmentation.

The cup (or disc) area is calculated as the number of pixels contained within the cup (or disc) segmentation. Maximum length measures are calculated as the maximum length of the row or column contained in the cup or disc. The length and area ratio measures of the optic disc, based on manually drawn contours of the cup and disc (by the ophthalmologist) show an increment in deformation of the ONH in the follow-up measures as listed in Tables I and II. The new computer generated volume measures (using semi-automatic segmentation) also follow the same trend, namely an increase in the deformation of the optic disc, in the same eye of patient one during 10 years of evaluation between 1989 and 1999.

TABLE I
Measures based on ophthalmologist's manual segmentation (MO)

| | Vertical | Vertical | Horiz. | Horiz. | | | | |
|---|---|---|---|---|---|---|---|---|
| Data | cup | disc | cup | disc | Cup | Disc | Cup | Disc |
| From | length | length | length | length | area | area | volume | volume |
| Patient | 196 | 280 | 185 | 261 | 27009 | 56250 | 234305 | 312619 |
| 1 1989 | | | | | | | | |
| Patient | 198 | 270 | 176 | 248 | 25959 | 52981 | 263573 | 351062 |
| 1 1993 | | | | | | | | |
| Patient 1 | 211 | 281 | 197 | 255 | 32866 | 55911 | 291897 | 350259 |
| 1996 | | | | | | | | |
| Patient 1 | 198 | 264 | 188 | 240 | 29002 | 49757 | 192314 | 206171 |
| 1999 | | | | | | | | |

TABLE II
Measures based on the computer generated segmentation (CG)

| | Vertical | Vertical | Horiz. | Horiz. | | | | |
|---|---|---|---|---|---|---|---|---|
| Data | cup | disc | cup | disc | Cup | Disc | Cup | Disc |
| From | length | length | length | length | area | area | volume | volume |
| Patient | 198 | 243 | 153 | 194 | 22792 | 34779 | 218818 | 283568 |
| 1 1989 | | | | | | | | |
| Patient | 209 | 251 | 185 | 247 | 30032 | 46515 | 2904001 | 345188 |
| 1 1993 | | | | | | | | |
| Patient 1 | 223 | 242 | 180 | 200 | 32110 | 39087 | 298944 | 331957 |
| 1996 | | | | | | | | |
| Patient 1 | 226 | 244 | 176 | 197 | 31001 | 38174 | 203727 | 215083 |
| 1999 | | | | | | | | |

Other measures from longitudinal data sets from a patient (patient one) are shown in Table III. Both manual (clinical) and computer generated 2-D and 3-D measures show a consistent increase in deformation of the ONH in the patient.

TABLE III
2-D and 3-D cup/disc ratios by computer generated and manual segmentations

| | | | Cup to | Cup to | | | | |
|---|---|---|---|---|---|---|---|---|
| | | | disc | disc | | | Cup to | Cup to |
| Data File | Cup to disc | Cup to disc | horizontal | horizontal | Cup to | Cup to | disc | disc |
| (patient | vertical | vertical | length | length | disc area | disc area | volume | volume |
| number-year | length ratio | length ratio | ratio | ratio | ratio | ratio | ratio | ratio |
| of study) | (CG) | (MO) | (CG) | (MO) | (CG) | (MO) | (CG) | (MO) |
| Patient 1 | 0.81 | 0.7 | 0.79 | 0.71 | 0.66 | 0.48 | 0.77 | 0.75 |
| 1989 | | | | | | | | |
| Patient 1 | 0.83 | 0.73 | 0.75 | 0.71 | 0.65 | 0.49 | 0.84 | 0.75 |
| 1993 | | | | | | | | |
| Patient 1 | 0.92 | 0.75 | 0.90 | 0.77 | 0.82 | 0.59 | 0.9 | 0.83 |
| 1996 | | | | | | | | |
| Patient 1 | 0.93 | 0.75 | 0.89 | 0.78 | 0.81 | 0.58 | 0.95 | 0.93 |
| 1999 | | | | | | | | |

| | | | | |
|---|---|---|---|---|
| Correlation | 0.91 | 0.96 | 0.99 | 0.91 |
| coefficient | | | | |
| between MO | | | | |
| and CG | | | | |

(MO) = Manually Segmented by the Ophthalmologist
(CG) = Computer Generated

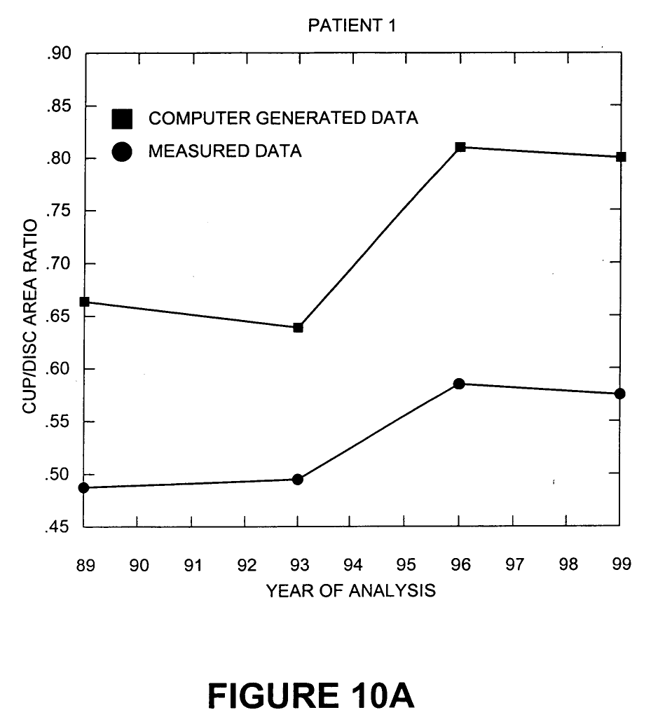

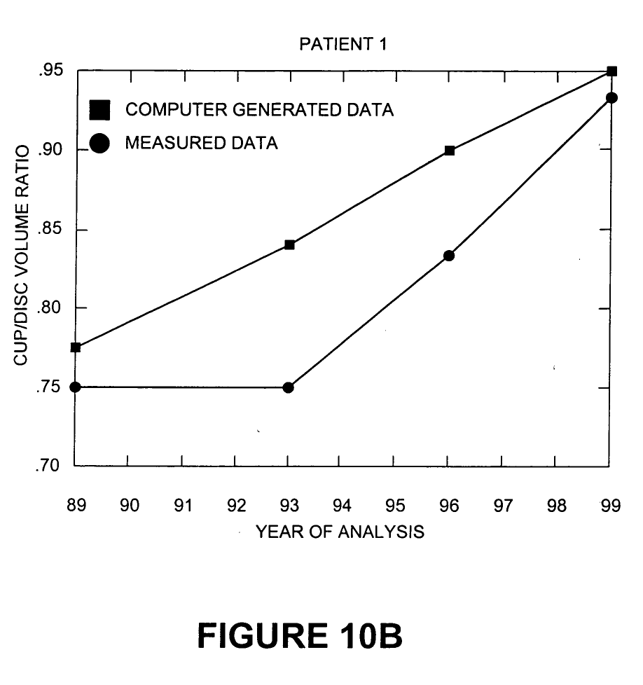

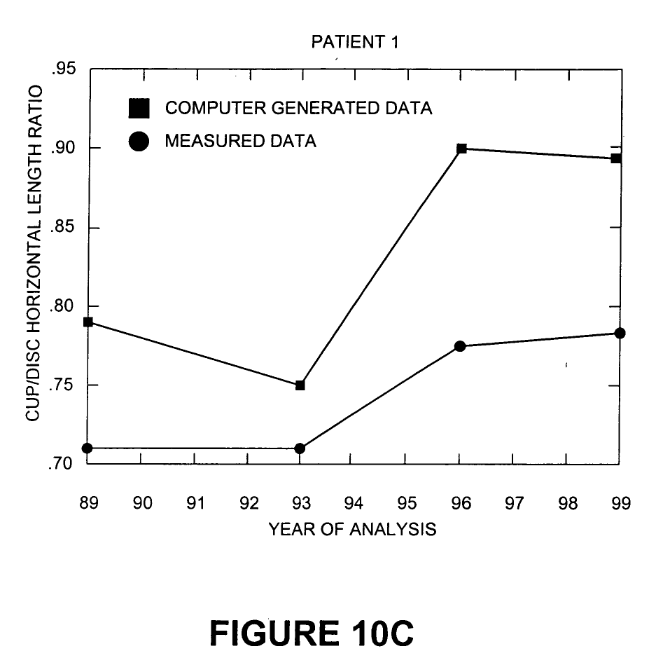

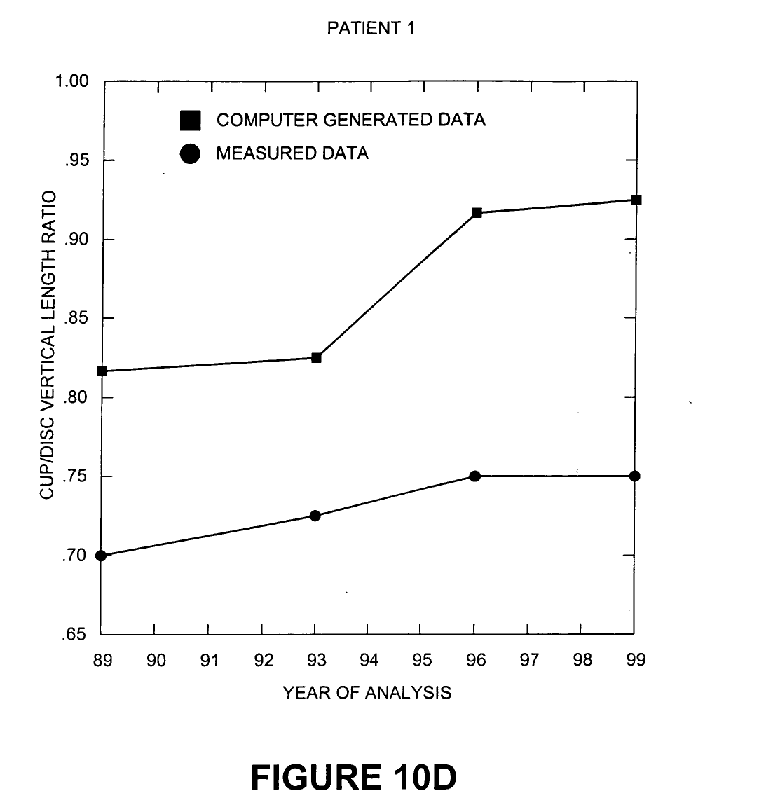

Results from clinical and computer generated measures for one patient over ten years are shown in plots of FIG. 10, suggesting good correlation for the new volume measures from clinical and computer generated cup/disc contours. Cup/disc ratios versus year of study for patient one. Plot through crosses shows the computer generated ratios. Plot passing through dots represent the ophthalmologist manual segmentation. A) shows area ratio, B) shows volume ratio, C) shows maximum horizontal length ratio and D) shows maximum vertical length ratio.

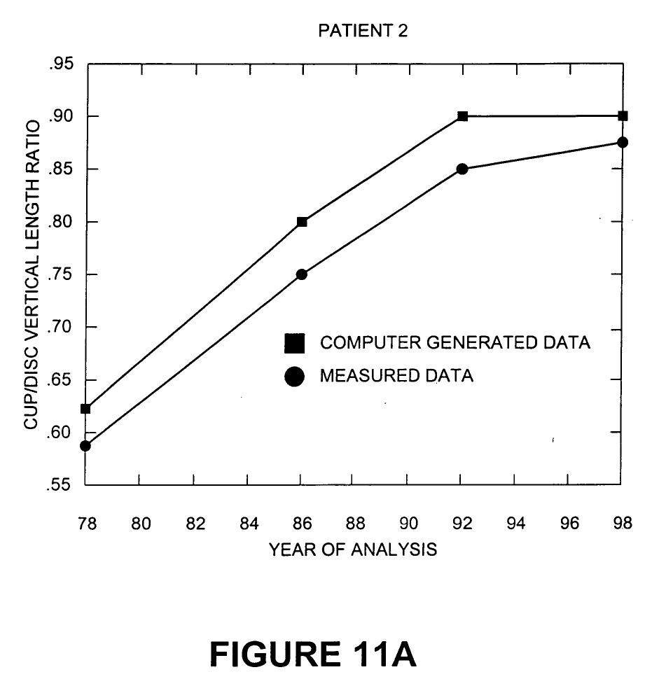

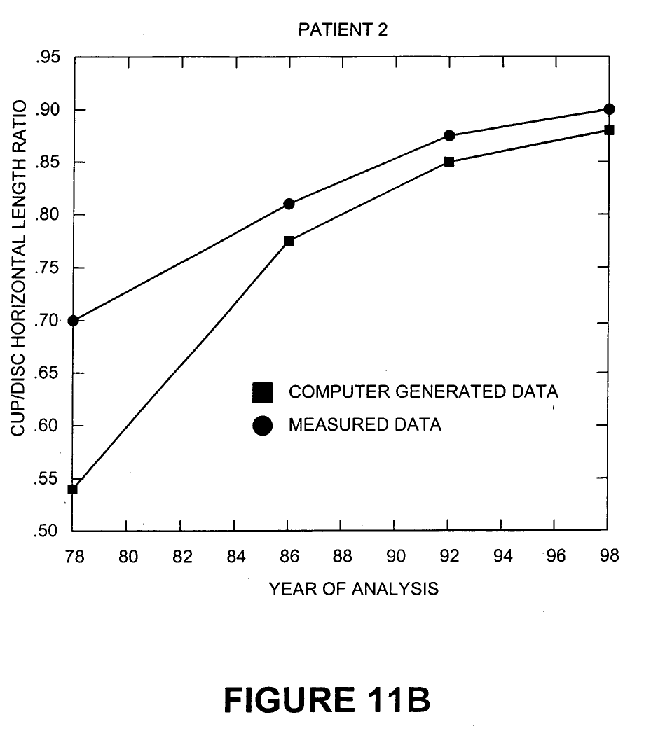

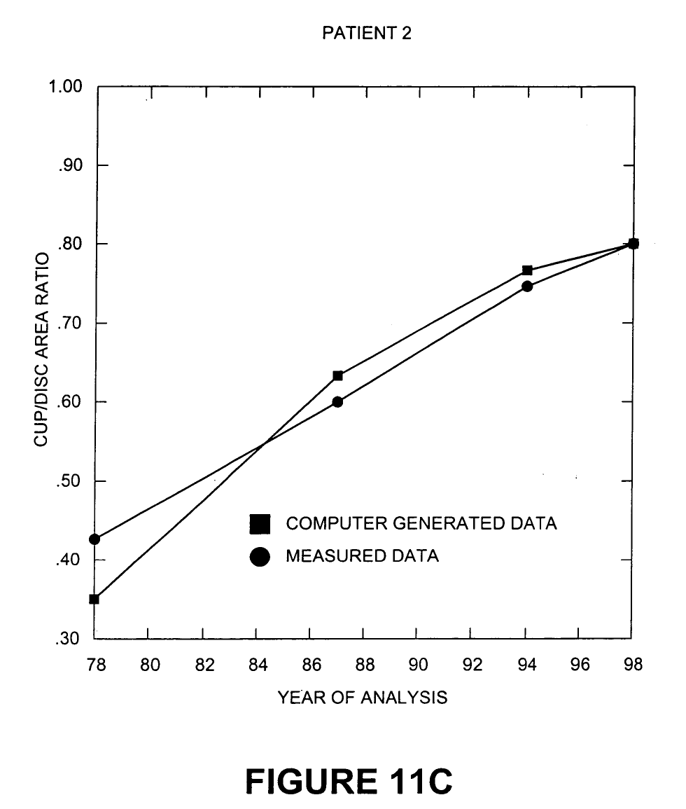

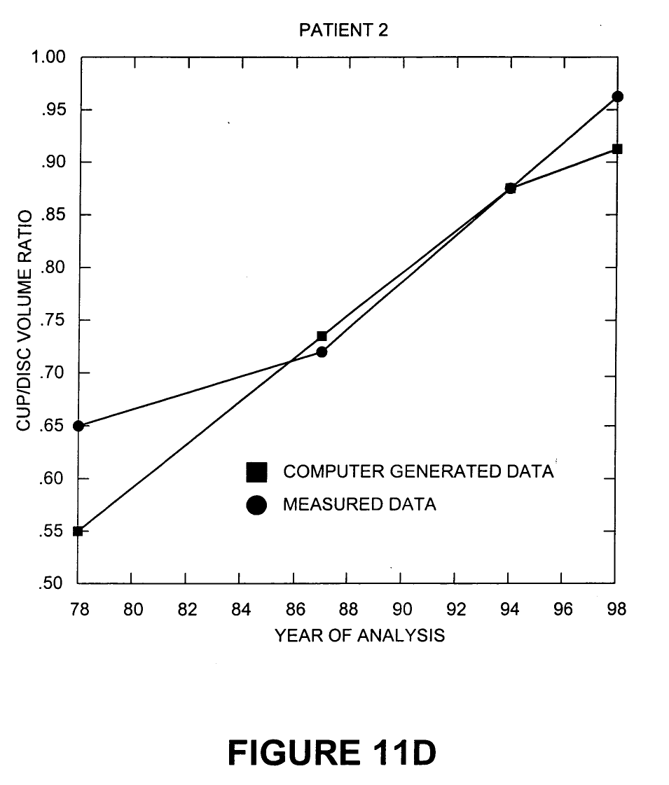

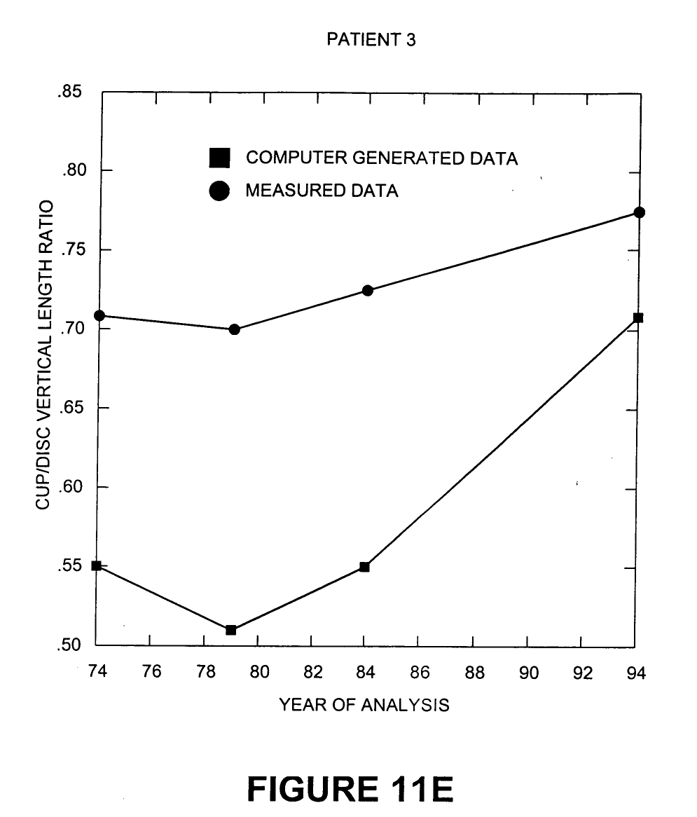

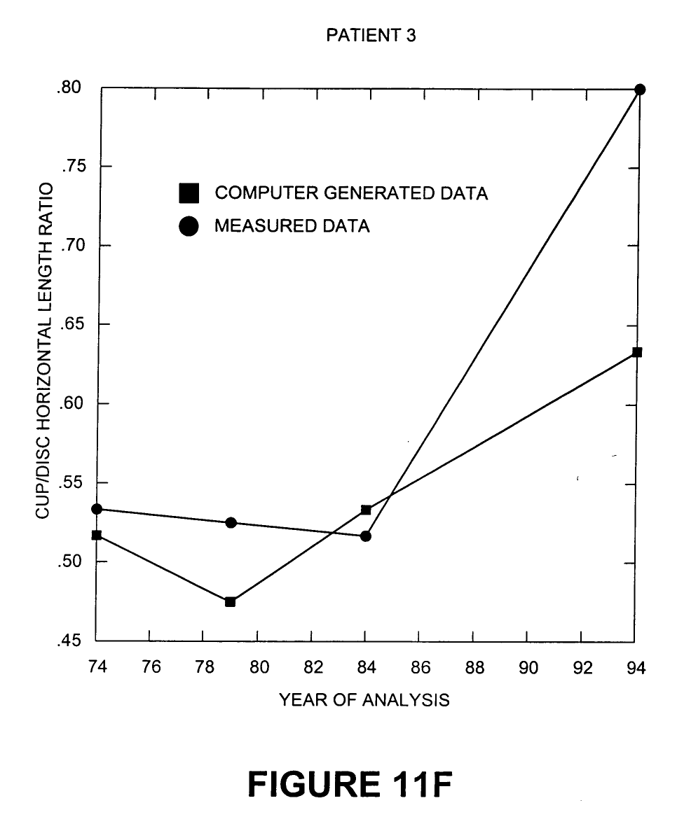

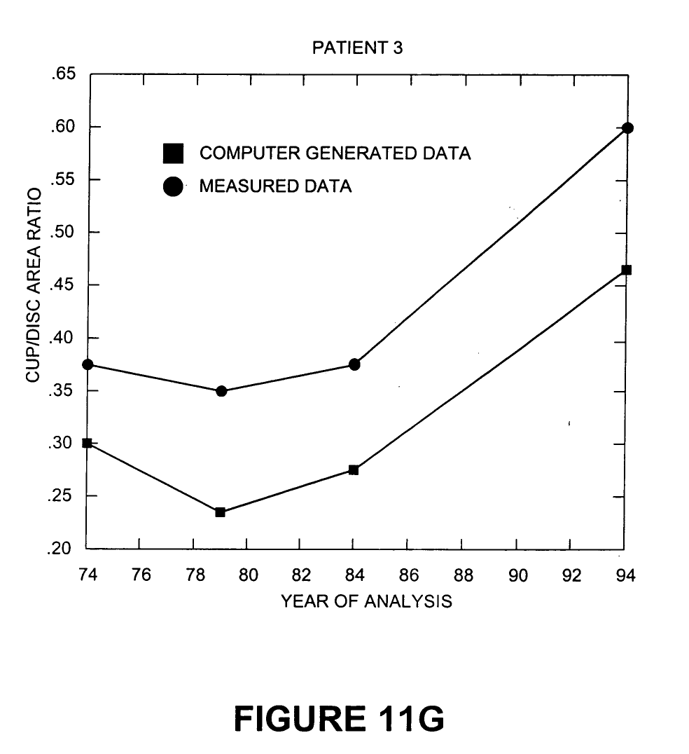

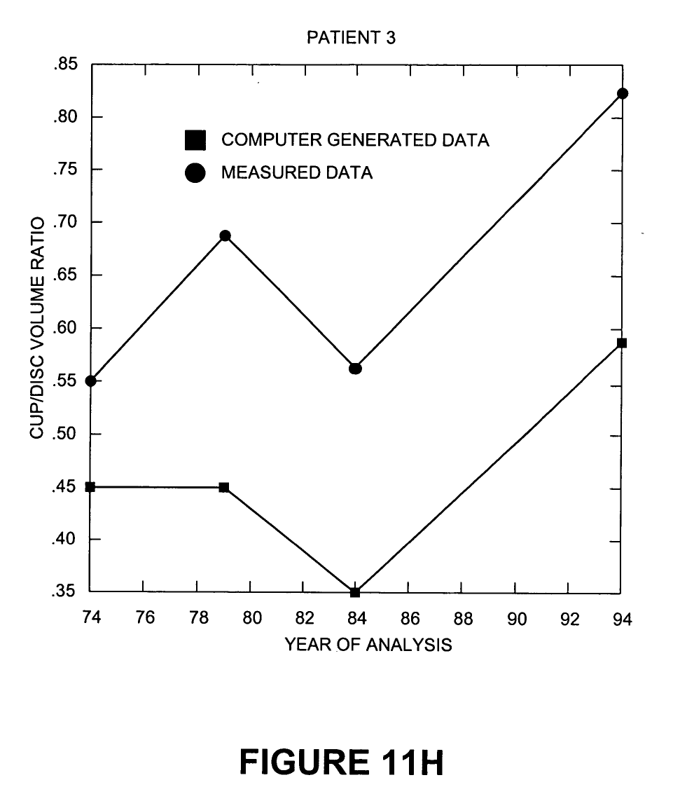

FIG. 11 shows a comparison of these measures for six patients. Cup/disc ratios versus year of study for three patients including patient one are plotted. First column (from left to right) represents maximum vertical length ratio, second column is maximum horizontal length ratio, third column is area ratio and fourth is volume ratio. First row of images represent analysis for patient one, second row for patient two and third row for patient three.

Table IV illustrates a comparison of correlation coefficients of various measures among each patient from longitudinal studies using four stereo pairs spanning ten to twenty years. Despite the challenges involved in 3-D surface recovery, the present invention results in consistent and more accurate measures of the ONH and shows significant correlation between the computer-generated measures and manual metrics commonly used in a clinical environment.

TABLE IV
Comparison of correlation coefficients of various measures among each
patient from longitudinal studies using four stereo pairs spanning ten to twenty years

| | | | Cup to | | |
|---|---|---|---|---|---|
| Longitudinal | | | disc | Cup to disc | Cup to disc |
| study | | Cup to disc | volume | horizontal | vertical |
| of patient | Spanning | area ratio | ratio | length | length |
| # | years | correlation | correlation | correlation | correlation |
| Patient 1 | 1989-1993-1996- | 0.99 | 0.91 | 0.96 | 0.91 |
| | 1999 | | | | |
| Patient 2 | 1978-1987-1994- | 0.98 | 0.96 | 0.99 | 0.99 |
| | 1998 | | | | |
| Patient 3 | 1974-1979-1984- | 0.99 | 0.86 | 0.93 | 0.96 |
| | 1994 | | | | |

A system and method for generating automated 3-D measures of optic disc deformation has now been illustrated. The system and method of the present invention, however, can be applied to assess topography of other retinal pathological structures such a macular edema, or edema in diabetic retinopathy that is currently evaluated by subjective impression of the topography by viewing of stereo pairs of the region concerned. It will be apparent to those skilled in the art that other types of equipment that record the necessary images may be employed and related statistical and analysis techniques may be used without departing from the scope of the invention as claimed.

What is claimed is:

1. A process for detecting optic disc deformation comprising:
obtaining a stereo image pair of an optic disc;
preprocessing the stereo image pair;
registering the stereo image pair;
extracting features from the stereo image pair;
finding course to fine disparities within the stereo image pair; and
obtaining a three dimensional representation of the optic disc.

2. The process for detecting optic disc deformation of claim 1 wherein:
obtaining a stereo image pair of an optic disc comprises obtaining the stereo image pair using a non-convergent imaging system.

3. The process for detecting optic disc deformation of claim 1 wherein:
pre-processing the stereo image pair comprises vertically registering the stereo pair;
dividing each of the images of the stereo pair into windows;
finding disparities by using a combination of the power cepstrum of the sum of corresponding windows of the stereo pair;
and cross-correlating the pixel values for both corresponding windows of the stereo pair.

4. The process for detecting optic disc deformation of claim 3 wherein vertically registering the stereo pair comprises:
finding the coarse to fine disparity between the stereo pair by computing the power cepstrum and cross-correlation for corresponding windows of the stereo pair.

5. The process for detecting optic disc deformation of claim 3 wherein vertically registering the stereo pair comprises:
calculating the frequency spectrum of each of the corresponding windows of the stereo pair

6. The process for detecting optic disc deformation of claim 1 wherein:
obtaining a three dimensional representation of the optic disc comprises obtaining a three dimensional representation of cupping of the optical disc.

7. A process for generating a three dimensional measure of optic disc deformation comprising:
obtaining a stereo image pair of an Optic Nerve Head (ONH);
identifying landmarks in each image of the stereo image pair;
identifying the disparity associated with depth of the stereo image;
aligning the images;
extracting binary features from the stereo images;
registering the stereo images in the vertical axis;
finding coarse to fine disparities of selected regions;
identifying the disparity for the finest ( highest resolution) window resulting in thereby creating a sparse disparity matrix;
smoothing the sparse disparity matrix using a cubic B-spline interpolation; and
superimposing the landmarks in the original stereo image pair image in the a 3-D representation of the Optical Nerve Head.

8. The process for generating a three dimensional measure of optic disc deformation of claim 7 wherein:
identifying the disparity associated with depth of the stereo image comprises triangulating corresponding points in the stereo image.

9. The process for generating a three dimensional measure of optic disc deformation of claim 7 wherein:
aligning the images comprises employing image matching strategies.

10. The process for generating a three dimensional measure of optic disc deformation of claim 7 wherein:
finding coarse to fine disparities of selected regions comprises computing a combination of the power cepstrum of the sum of corresponding windows and cross-correlation along a range of pixels.
